(19) United States
(12) Patent Application Publication
(10) Pub. No.: US 2005/0037001 A1
(43) Pub. Date: Feb. 17, 2005

(54) APC targeting conjugate, an antigen-presenting cell contacted with such conjugate, their medical use, and methods of production

(76) Inventors: Wilfred Thomas Vincent Germeraad, Gronsveld (NL); Ton Logtenberg, Driebergen (NL); Annemarie Nicolette Lekkerkerker, Utrecht (NL)

Correspondence Address: TRASK BRITT, P.O. BOX 2550, SALT LAKE CITY, UT, 84110, US

(21) Appl. No.: 10/856,272

(22) Filed: May 28, 2004

Related U.S. Application Data
(63) Continuation of application No. PCT/EP02/13681, filed on Nov. 29, 2002.
(63) Continuation-in-part of application No. PCT/EP01/14255, filed on Nov. 30, 2001.

(30) Foreign Application Priority Data
Dec. 19, 2001 (EP) EP01204997.9

(51) Int. Cl. A61K 39/395

(52) U.S. Cl. 424/141.1; 424/144.1

(57) ABSTRACT

A conjugate for targeting antigen-presenting cells, including at least one antigenic moiety conjugated to a targeting moiety that is capable of binding to a cell surface structure of an antigen-presenting cell, wherein the conjugate is capable of being internalized and processed by the antigen-presenting cells such as to cause processed antigenic moiety fragments thereof to be presented via MHC class I and MHC class II molecules of the antigen-presenting cell, to a nucleic acid sequence including, a nucleic acid sequence encoding the antigenic moiety and a nucleic acid sequence encoding the targeting moiety, to a host cell to a method for producing a conjugate, and for generating an antigen-presenting cell capable of eliciting an immune response to such antigen-presenting cell, to a pharmaceutical composition comprising a conjugate or an antigen-presenting cell and their use for vaccination, and as a medicament.

### CROSS-REFERENCE TO RELATED APPLICATIONS

This application is a continuation of PCT International Patent Application PCT/EP02/13681, filed Nov. 29, 2002, published in English as WO 03/046012 on Jun. 5, 2003, which is a continuation-in-part of PCT International Patent Application PCT/EP01/14255, filed Nov. 30, 2001, published in English as WO 03/046011 on Jun. 5, 2003, and claims the benefit under 35 U.S.C. § 119 of European Application No. EP01204997.9, filed Dec. 19, 2001, the entirety of which are incorporated by reference.

### TECHNICAL FIELD

The present invention relates generally to biotechnology, and more particularly to a conjugate for targeting antigenic material to antigen-presenting cells, pharmaceutical preparations containing conjugates or antigen-presenting cells (APC) so produced, and to conjugates or APCs for use in medical treatment. The invention further relates to the use of such conjugates to manufacture medicaments for the prophylactic and/or therapeutic treatment of humans or animals to treat or prevent disease or discomfort.

### BACKGROUND

Conjugates for targeting antigenic moieties to antigen-presenting cells as such are known. They typically comprise at least one antigenic moiety conjugated to a targeting moiety. The targeting moiety determines the type of antigen-presenting cell that is targeted. The antigenic moiety is the part of the conjugate which, after internalization, is processed by the machinery of the antigen-presenting cell, and fragments thereof are presented via major histocompatibility complex (MHC) class I or MHC class II molecules. This results in cytolitic T-lymphocyte (CTL) activation or T-helper (Th) cell activation, respectively, thereby inducing an antigenic moiety-specific immune response which is either of the cytotoxic T-cell type or of the humoral type. CTL activation is essential for killing tumor cells. For full induction of CTL, cytokines produced by Th1-cells are necessary. For a production of the antibodies by B-cells, Th2-cells are necessary for stimulating the B-cells.

Wallace et al., 2001, reported that targeting anti-Fc gamma R1 to a myeloid cell line using Fab-PSA (prostate-specific antigen) resulted in a MHC class I associated presentation, followed by killing of the myeloid cell line. This method will suffer from the disadvantage that the distribution pattern of the Fc gamma receptor is not restricted to professional antigen-presenting cells.

Articles by Amigorena et al., 1999; and Machy et al., 2000, reported that with liposomal formulations containing antigen, MHC class I and MHC class II presentation may be obtained. However, this means of delivery of antigen is not specific for professional antigen-presenting cells and could result in the antigenic moieties to end up in various other tissues of the human or animal body, with the associated risk of causing potential side effects.

One particular type of professional antigen-presenting cells is dendritic cells. Lekkerkerker and Logtenberg 1999 described a series of scFvs monoclonal antibody fragments which recognize human dendritic cell sub-populations. The authors hypothesized that converting these scFv-antibody fragments into complete human antibodies and fusing them to an antigen may be used for targeted delivery of antigens to sub-populations of dendritic cells for therapeutic applications; however, no results have been shown.

Thus, although initial promising results have been obtained in the art, there remains a need for methods of targeting to and presenting antigens by antigen-presenting cells that provide both a complete immune response and specificity in terms of targeting to professional antigen-presenting cells.

### SUMMARY OF THE INVENTION

According to the present invention, a conjugate for targeting an antigen-presenting cell is provided, comprising: at least one antigenic moiety conjugated to a targeting moiety that is capable of binding to a cell surface structure of an antigen-presenting cell and, upon binding, inducing a CTL response and a T-helper response.

In particular, according to the present invention, a conjugate for targeting antigen-presenting cells is provided, comprising at least one antigenic moiety conjugated to a targeting moiety that is capable of binding to a cell surface structure of an antigen-presenting cell, wherein the conjugate is capable of being internalized and processed by the antigen-presenting cell such as to cause processed antigenic moiety fragments thereof to be presented via MHC class I and MHC class II molecules of the antigen-presenting cell.

The invention also provides a nucleic acid sequence comprising a nucleic acid sequence encoding the antigenic moiety capable of inducing a CTL response and a T helper response upon binding to an antigen-presenting cell, and a nucleic acid encoding a targeting moiety that is capable of binding to a cell surface structure of an antigen-presenting cell. The invention also provides a nucleic acid encoding an antigenic moiety capable of being internalized and processed by the antigen-presenting cells such as to cause processed antigenic moiety fragments thereof to be presented via MHC class I and MHC class II molecules of the antigen-presenting cell.

The invention further provides a host cell transformed or transfected using a nucleic acid sequence encoding the antigenic moiety capable of inducing a CTL response and a T helper response upon binding to an antigen-presenting cell and/or being internalized and processed by the antigen-presenting cells such as to cause processed antigenic moiety fragments thereof to be presented via MHC class I and MHC class II molecules of the antigen-presenting cell, and a nucleic acid sequence encoding a targeting moiety that is capable of binding to a cell surface structure of an antigen-presenting cell. Optionally, the nucleic acid may be an expression vector, wherein the expression vector may comprise the nucleic acid sequence operably linked to an expression sequences for an antigen-presenting cell, an expression sequence adapted for expression in mammalian cells, such as PER.C6, a promoter obtainable from hCMV, and/or a polyA signal obtainable from the bovine growth hormone.

According to a further aspect the invention, provided is a method for producing a conjugate comprising antigenic moiety capable of inducing a CTL response and a T helper response upon binding to an antigen-presenting cell and/or being internalized and processed by the antigen-presenting cells such as to cause processed antigenic moiety fragments thereof to be presented via MHC class I and MHC class II molecules of the antigen-presenting cell, conjugated to a targeting moiety that is capable of binding to a cell surface structure of an antigen-presenting cell, the method comprising culturing host cells under conditions allowing expression of a nucleic acid of the invention encoding the conjugate, whereby the conjugate is formed, and isolating the conjugate from the cells and/or culture medium.

A method for generating an antigen-presenting cell capable of eliciting an immune response via MHC class I and MHC class II presentation of processed antigen fragments is furthermore provided, the method comprising contacting an antigen-presenting cell with a conjugate according to the invention.

The invention further provides an antigen-presenting cell capable of eliciting an immune response via MHC class I and MHC class II presentation of processed antigen fragments, the antigen-presenting cell obtainable by contacting an antigen-presenting cell with a conjugate according to the invention, as well as the use of a conjugate according to the invention or an antigen-presenting cell according to the invention for prophylactic or therapeutic vaccination.

A conjugate according to the invention or an antigen-presenting cell according to the invention, such as by contacting an antigen-presenting cell with a conjugate of the invention, for use as a medicament is provided.

The invention also provides a conjugate according to the inventior or an antigen-presenting cell produced according to the invention for use in the prevention, retardation and treatment of a disease selected from the group consisting of Alzheimer, atherosclerosis, cancer, diabetes, HIV-seropositivity, AIDS, Hepatitis, and the like.

### DESCRIPTION OF THE FIGURES

FIG. 1 is the amino acid sequence of VH region of MatDC16;

FIG. 2 depictspPicZFVH-S1/23-hgp100;

FIG. 3 is the Northern blot analysis of transient transfections: 1) mock; 2) IgG4 MatDC16; 3) IgG4 MatDC16-MAGE-1; 4) MAGE-1, probed with a MAGE-1 probe;

FIG. 4 is the Western blot analysis of the purified IgG4 MatDC16-MAGE-1 (lane 1) and IgG4 MatDC16 (lane 2) with mouse-anti-MAGE-1 and RAMPO, visualized by ECL;

FIG. 5 shows the flow cytometric assay detecting both ends of different IgG4 MAGE-AL fusion constructs. The example shows binding of antibodies to monocytes, gate based on FSC/SSC. Transparent histogram: IgG4 MAGE-1, followed by mouse anti-MAGE-1 MoAb/goat-anti-mouse Ig-PE; grey histogram: mouse anti-MAGE-1 MoAb/goat-anti-mouse Ig-PE; and

FIG. 6 illustrates the comparative ability of tumor Ag presentation by immature DC incubated with fusion Abs. Immature DC (10⁵) derived from an HLA-A1+/HLA-DR1301+ donor were incubated with fusion Abs (10 nM or 100 nM). Before adding the proteins, the DC were incubated with 20% human serum for 30 minutes on ice to block Fc gamma receptors. Either after (A) 24 or (B) 48 hours of incubation, cocultures of DC (15000) with (I) CTL anti-MAGE-A1.A1 or (II) T_{H} anti-MAGE-A1.DR1301 (5000) were set up. Activation was assessed as IFN-g release at 24 hours. Data are presented as picograms of IFN-gamma released/5×10³/ml/24 hours (mean±SD of triplicate cultures).

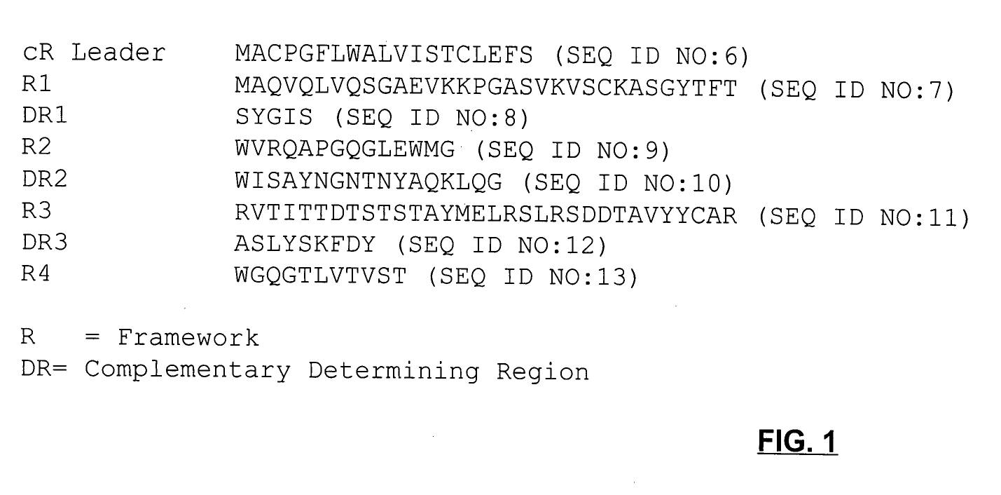

### DETAILED DESCRIPTION OF THE INVENTION

The present invention provides, by way of targeting a conjugate comprising an antigen-determining moiety and a targeting moiety with specificity for antigen-presenting cells, for the targeted delivery, up-take, processing and presentation of antigenic material by antigen-presenting cells, whereby antigenic fragments of the conjugate are presented via MHC class I and class II molecules causing, by inducing a CTL response and a T-helper response, an effective induction of all arms of the adaptive immune system.

If the antigenic moiety is of parasitic, fungal, bacterial, viral or autologous (tumor) origin, then, due to the specific immune response, the conjugate functions as an anti-parasitic, anti-fungal, anti-bacterial, anti-viral or anti-tumor agent, respectively.

The antigen-presenting cell which presents antigenic moiety fragments may be generated in vitro or in vivo. This means that both the conjugate and antigen-presenting cell contacted with the conjugate in vitro or in vivo is suitable for use in prophylactic or therapeutic vaccination and as a medicament for the antigenic moiety-related diseases. A full immune response is obtained when an antigenic moiety is taken up, processed and presented by the professional antigen-presenting cell.

In cancer therapy, immunotherapy is an option for local tumors and metastasis after surgery. Immunotherapy requires a humoral response and a cellular response. For a cellular response, both a CTL activation (class I restricted) and Th cell activation (class II restriction) are necessary. T-helper responses lead to better CTL activity (Th1) as well as humoral responses (Th2).

The present invention is based on the finding that a conjugate comprising an antigenic moiety conjugated to a targeting moiety results after internalization and processing by the antigen-presenting cell in a presentation of antigenic moiety fragments by both MHC class I and MHC class II molecules and in an antigenic moiety-specific adaptive immune response.

The conjugate for targeting antigen-presenting cells comprises at least one antigenic moiety conjugated to a targeting moiety. The targeting moiety specifically directs the conjugate to a cell surface structure of the antigen-presenting cell.

The antigen-presenting cell may be a B-cell, a monocyte, or a dendritic cell. These cells may originate from blood, tonsil, synovial fluid or bone marrow. In blood, the B-cells may be CD19⁺ cells; in tonsil, CD19⁺ B-cells. The monocytes may be, in blood, CD14⁺ monocytes and in bone marrow, CD14⁻ monocytes. The dendritic cells may be dendritic cells at any stage of maturation. Particular groups are CD33⁻ CD14⁻ dendritic cells and CD33^{dim} CD16⁻ dendritic cells. Many different types of dendritic cells exist within the organism, in particular, at interfaces with the outside environment in order to pick up invaders. However, all organs contain dendritic cells to pick up endogenous signals like those of tumor-derived antigens. In blood, at least two different phenotypically and functionally distinct sub-populations of dendritic cells can be found. The first population comprises CD33^{dim}CD14⁻CD16⁻ cells (Thomas et al., 1993; Thomas and Lipsky, 1994), by others called the CD11c⁻ DC lacking lineage-specific markers (Lin−; O'Doherty et al., 1994). The Lin⁻HLA-DR⁺ cells express high levels of CD123 (Olweus et al., 1997). These DCs are thought to represent a precursor population capable of taking up and processing antigen but are less efficient to present the resulting peptides to T-cells. They phenotypically resemble a DC precursor population that can be found in the paracortex of lymphoid tissues (Grouard et al., 1997). The second blood DC population is characterized by CD33⁺CD14⁻CD16⁻, or can be identified as Lin⁻CD11c⁺. These latter cells are considered more mature as they can better present antigen than the precursor DC population (Thomas and Lipsky, 1994). Similar cells can been found in germinal centers of follicles in lymphoid tissue (Grouard et al., 1996).

Antigen-presenting cells have, in principle, the capability, after binding of the conjugate to a particular cell surface structure, of internalization and processing of the antigenic moiety of the conjugate and presentation of processed antigenic moiety fragments via both MHC class I and MHC class II molecules. However, it is not known in detail what determines whether antigen fragments are presented via MHC class I or II, or both.

The targeting moiety is capable of binding to a cell surface structure of the antigen-presenting cell via a specific immunological binding. The targeting moiety may be selected from an immunoglobulin or a fragment thereof, such as a scFv fragment, Fab fragment, or F(ab)2 fragment. Decisive is a specific binding of the targeting moiety to a cell surface structure of a particular antigen-presenting cell. Preferably, the targeting moiety is of a monoclonal nature, which improves its selective binding to its target cell surface structure. Preferably, the targeting moiety is humanized (or human) in order to reduce its inherent antigenic properties. In order to improve the binding of the targeting moiety and, thereby, the conjugate to a cell surface structure of the antigen-presenting cell, it is preferred that the targeting moiety is in a bivalent or polyvalent form, in particular, bivalent or polyvalent forms of immunoglobulins or fragments thereof. According to one preferred embodiment, the targeting moiety comprises an IgG4 subtype immunoglobulin.

A particular group of targeting moieties according to the invention is formed by monoclonal phage antibodies, such as MatDC11, MatDC16, MatDC27, MatDC51 and MatDC64. MatDC16 shows preferred targeting properties for mature and precursor dendritic cells and monocytes, such as CD14+monocytes, CD19+B-cells in blood, for a subpopulation of granulocytes and CD19⁺ B-cells in tonsil, for mature and precursor dendritic cells and CD33⁺ CD14⁺ monocytes in synovial fluid, and for CD14⁺ monocytes and CD15⁺ and CD34⁺-cells in bone marrow. The sequence of the MatDC16 VH region comprises amino acids 1 to about 743 of SEQ ID NO:14. The CDR3 region of the VH region is ASLYSKFDY (SEQ ID NO:12). The VL chain is of the Vκ2 subtype. Obviously, encompassed by the invention are affinity-matured mutants thereof. Affinity-matured mutants may be obtained using techniques well known in the art, such as the polymerase chain reaction using primers that introduce modifications with respect to the original complementarity-determining regions of the targeting moiety. Such modifications may be amino acid substitutions, deletions and/or additions within one or more CDRs of the targeting moiety. The method of modifying the binding specificity or affinity of the targeting moiety is not critical to the instant invention.

The conjugate comprises at least one antigenic moiety. It may be a peptide, polypeptide, protein, glycoprotein, lipoprotein, and/or a derivative or fragment thereof. A derivative or fragment thereof means a part of a processed version of the antigenic moiety in which is preserved the particular antigenic reactive part or epitope. It is noted that many antigenic moieties comprise more than one epitope. This antigenic moiety may be of parasitic origin, such as plasmodium vivax merozoite surface protein 1, acidic basic repeat antigen of plasmodium falciparum, or plasmodium falciparum liver stage antigen-3. Examples of antigenic moieties from fungal origin are members of the aspartyl proteinase family, 65 kDa mannoprotein antigen and yeast-killer toxin receptor. Antigenic moieties of bacterial origin may be exemplified as those relating to the diseases pneumonia, meningitis and bacteremia. Examples of antigenic moieties from viral origins are env, gag, S major env, preS2 middle, and G2Na. Antigenic moieties of autologous origin, such as tumors, are exemplified as MAGE, such as MAGE-1 (melanoma-associated antigen), MAGE-3, gp100, Muc-1, Her2/neu, PSA, PSMA and CEA. Those of skill in the art will recognize that the invention may be practiced using other tumor-associated antigens than those mentioned here, or even any disease-associated antigen, for that matter. MAGE-1 is a well-characterized tumor antigen already used in clinical trials (Rosenberg et al., 1998; Rosenberg et al., 1999; Nestle et al., 1999), was chosen for this study. In the past years, melanoma-specific CTL clones have served as tools to identify genes that code for tumor antigens (Boon et al., 1996). The MAGE gene family includes at least 17 related genes, namely, MAGE-AL to A12, MAGE-B1 to B4, and MAGE-C1. The MAGE genes are expressed by tumors of various histological types; however, they are silent in normal cells, with the exception of male germ-line cells that do not carry MHC class I molecules and are, therefore, unable to present antigens to CTL. Hence, antigens encoded by MAGE-A, -B, -C genes should be strictly tumor-specific. Because the MAGE antigens are shared by many tumors and on account of their strict tumor specificity, they are of particular interest for cancer immunotherapy. Gene MAGE-A1 was isolated because it encoded an antigen presented on HLA-A1 molecules to autologous CTL of a melanoma patient (van der Bruggen et al., 1991). A preferred targeting moiety is formed by MatDC16 (SEQ ID NO:21), which is a monoclonal phage antibody binding to blood CD33⁺CD14⁻CD16⁻ mature dendritic cells.

It is not required that the antigenic moiety of the conjugate is in the form of an amino acid sequence. In another embodiment of the conjugate according to the invention, the antigenic moiety may be in the form of a nucleic acid sequence which encodes the antigenic peptide, polypeptide or protein, or a precursor thereof. In this embodiment, the antigenic moiety is preferably in the form of an expression vector. When the conjugate is targeting a mammalian antigen-presenting cell, then it is preferred to use a vector which is adapted for expression in mammalian cells, more in particular, human cells. Nucleic acid sequences which may be used to express gene sequences in mammalian cells, such as human dendritic cells, are well known to those skilled in the art. In a preferred embodiment, the antigenic moiety in the form of a nucleic acid sequence is conjugated to a targeting moiety that has the form of an immunoglobulin or fragment thereof.

When the antigenic moiety is in the form of an expression vector, for expression, it is preferred that the nucleic acid sequence encoding the antigenic moiety is operably linked with expression sequences for the antigen-presenting cell for which the conjugate targets. For an optimal expression in the antigen-presenting cell, it is preferred that the expression sequence comprises a promoter obtainable from hCMV and/or a polyA signal obtainable from the bovine growth hormone.

The conjugate may be formed by conjugating a particular antigenic moiety to a particular targeting moiety. Conjugation may be obtained by any chemical or affinity conjugation mechanism. Conjugation may be between biotin-streptavidin complexes or via polymers, such as poly-L-lysine(PLL) and polyethylenimine (PEI), or may be Ni²⁺-6× Histidine tag-binding, but not limited to these forms.

It is preferred to produce the conjugate in a host cell which is transformed or transfected with a nucleic acid sequence encoding the antigenic moiety and the targeting moiety as a single polypeptide chain, although other forms of conjugation, such as via sulfur bridges, are contemplated. The host cells are cultured in a culture medium under conditions allowing expression of the nucleic acid encoding the conjugate. The conjugate formed is isolated either from the cells or from the culture medium, or from both. The host cells are preferably PER.C6-TM cells. It is to be understood that the term “host cells” also refers to host cells present in vivo. The in vivo host cells can be employed to produce the conjugate encoded by the nucleic acid, and thereby function as an in vivo platform.

Contact of the conjugate and the antigen-presenting cell may be carried out in vivo or in vitro. In vitro, a particular type of antigen-presenting cell, after its isolation and production is contacted with the conjugate in a contact medium. Due to the contact, the antigen-presenting cells, after internalization and processing of the antigen moiety, will be able to present at their surface via MHC class I and MHC class II molecules antigenic fragments of the antigenic moiety of the conjugate. These antigen-presenting cells are harvested from the medium and administered to the patient where presentation takes place of the processed peptides by the APC to CTLs and T-helper cells in vivo. These harvested antigen-presenting cells may be used for prophylactic and/or therapeutic vaccination, as a medicament or as an anti-parasitic, anti-fungal, anti-bacterial, anti-viral or anti-tumor agent. They may also be used in immunotherapy.

The contact of the conjugate with the antigen-presenting cells may also be carried out in vivo. The organism in which the antigen-presenting cells occur is exposed to the conjugate, for example, via subcutaneous, intradermal, intramuscular, colorectal, mucosal or intravenous injection of the conjugate. The targeting moiety of the conjugate directs the conjugate to the particular antigen-presenting cells and after target binding, the antigenic moiety of the conjugate is internalized, processed and subsequently fragments thereof presented via MHC class I and MHC class II molecules on the surface of the antigen-presenting cell targeted. Antigen-presenting cells presenting in the organism function as a therapeutic agent for vaccination or medication for the above-exemplified therapeutic uses. In vivo contact of the antigen-presenting cell with the conjugate provides the further advantage of an extended presentation by the antigen-presenting cell of the fragment of the antigenic moiety. This allows a more stable immunological synapse to form (Lanzavecchia and Sallusto, 2001). The in vivo generation of antigen-presenting cells eliciting immune responses via MHC class I and MHC class II presentation of the antigen fragments may also occur with a conjugate comprising the antigenic moiety in its nucleic acid encoding format.

The conjugate according to the invention and the antigen-presenting cells eliciting via MHC class I and MHC class II presentation of processed antigen fragments of the conjugate demonstrate a new antigen (fragment) delivery and a more effective immune response, which makes these conjugates and conjugate-activated antigen-presenting cells optimal agents for vaccines and immunotherapies. They are suitable for use in the treatment of, in particular, autologous and infectious diseases, such as Alzheimer, atherosclerosis, cancer, diabetes, AIDS, hepatitis and the like.

Hereafter the present invention will be further illustrated by the use of a particular conjugate. As the antigenic moiety, this conjugate comprises the MAGE-1 antigen (starting at approximately amino acid 744 of SEQ ID NO:21). Using the MAbstract-TM procedure, particular monoclonal phage antibodies recognizing mature dendritic cells have been isolated. The present invention is not restricted to this isolation procedure or specific antigen-presenting cells. The procedure may also be applied to in vitro monocyte-derived dendritic cells and any in vivo DC population from any particular organ as can be defined with specific antibodies (such as tonsil, skin, lung, liver, thymus).

Hereinafter, the invention is illustrated in greater detail by the production of particular DC-targeting conjugates delivered to antigen-presenting cells that present fragments of the tumor antigen MAGE-1 via MHC class I and MHC class II molecules on their surface, without considering the invention to be restricted thereto.

### Deposits

MatDC16, as well as MatDC11, MatDC27, MatDC51 and MatDC64 have been deposited according to the Budapest Treaty at the ECACC on Dec. 4, 2001, under the following accession numbers: 01120417 (including SEQ ID NO:21), 01120416, 01120418, 01120419 and 01120420, respectively.
cDNA encoding the VH and VL of the deposited MatDC16, as well as MatDC11, MatDC27, MatDC27 and MatDC64 are present in the pHEN vector as a scFv fragment fused to a Myc-tag for detection with the monoclonal antibody 9E10 and a 6×HIS-tag allowing later affinity purification of a produced scFv. These vectors are in the E. coli strain XL-1 blue and can be rescued by growing them on TY-agar plates containing ampicillin and tetracycline according to methods known in the art. The VH cDNA can be removed from the plasmid by NcoI and XhoI digestion, and the VL by SacI and NotI restriction enzymes.

### EXAMPLES

1. Phage Display Library

Previously, a large phage antibody display library of human single chain antibody fragments was constructed (de Kruif et al., 1995). The library consists of a combination of 49 germline VH genes fused with ˜10⁸ synthetic heavy chain CDR3 regions and seven light chains. The CDR3 regions vary in length between 6 and 15 amino acids. The light chains are encoded by members of the Vκ1 to Vκ4 and Vλ1 to Vλ3 families. The final library size consists of about 4×10⁸ individual clones.

2. Selection of Phage Antibodies by Cell Sorting

Eighty ml of human blood was diluted 1:1 with RPMI 1640 medium and layered on top of a Ficoll cushion. After 20 minutes of centrifugation, the interface containing peripheral blood mononuclear cells (PBMC) was recovered. Forty×10⁶ PBMCs and 2-amino-ethylisothio-uroniyum bromide hydrobromide- (AET-) treated sheep red blood cells were pelleted and incubated on ice for one hour. This resulted in the formation of T-cell-SRBC rosettes that could be removed after another centrifugation over a Ficoll cushion. The phage antibody library, containing approximately 10¹³ phage particles per ml, was blocked for 15 minutes in 250 μl of PBS/5% (w/v) milk powder. Subsequently, the obtained cell mixture, consisting mainly of monocytes, B-lymphocytes and the described dendritic cells, were added to the blocked phages and the mixture was slowly rotated overnight at 4° C.

The next day, the cells were washed twice with ice-cold PBS/1% (w/v) BSA and were stained with 20 μl PE-conjugated anti-CD33 antibody and 20 μl FITC-conjugated anti-CD14 antibody to visualize different cell populations on a flow cytometer. After 20 minutes incubation on ice, the cells were washed once with PBS/1% BSA and resuspended in 4 ml of PBS/1% BSA. Cell sorting was performed on a FACStar^{PLUS} fluorescence-activated cell sorter with the gates set around the CD33⁻CD14⁻ mature DCs. For this cell population, 10⁴ to 10⁵ cells with phages still attached were sorted.

To elute specifically bound phages, the cells were pelleted and transferred in a volume of 100 μl of M-PBS to a 15 ml tube containing 150 μl of sodium citrate (pH 2.5). After five minutes, the pH was neutralized by adding 125 μl of 1 M Tris HCl buffer (pH 7.4). Finally, 3 ml of 2TY medium and 3 ml of log phase Escherichia coli XL-1 blue were added. Infection was allowed to proceed for 30 minutes at 37° C. Bacteria were centrifuged at 2,200×g for 20 minutes, suspended in 0.5 ml of 2TY, and plated on agar plates containing 25 μg/ml tetracycline, 100 μg/ml ampicillin, and 5% glucose (TAG). After overnight culture at 37° C., plates were scraped and bacteria were frozen in stock vials or used to prepare the next restricted library using a helper phage. After the first round of selection 1×10⁵ colonies were obtained for the selection with mature DCs.

The selection round described above was repeated two more times and after the third round, bacteria were seeded in the proper dilution allowing isolation of single colonies. These clones were individually grown and rescued with helper phage to prepare monoclonal phage solutions. Every clone was then tested on the original population for specific binding to the DC population.

3. Identification of Phages Selected for Binding on Mature DC

Forty-two out of 90 Monoclonal Phage antibodies (MoPhab) derived from the selection on the CD33⁺ CD14⁻ cells bound exclusively to the CD33⁺ cells or displayed additional binding to small subpopulations of CD33⁻ cells. From these 42 clones, plasmid DNA was isolated from the bacteria using the Qiagen miniprep kit. The scFv DNA coding region was amplified with primers: LMB3 (5′-CAGGAAACAGCTATGAC) (SEQ ID NO:1) and fd-SEQ1 (5′GAATTTTCTGTATGAGG) (SEQ ID NO:2) under the following conditions: one minute denaturing at 94° C., one minute annealing at 55° C. and two minutes extension at 72° C. The resulting PCR product was digested with BstNI for one hour at 37° C. resulting in the appearance of various bands of different length. On the basis of the BstNI fingerprint, indicating differences in identity of individual phages, five MoPhabs, namely, MatDC11, MatDC16, MatDC27, MatDC51 and MatDC64, were propagated for further analysis. From all five clones, the sequence of the VH and VL were determined using primer M13REV (5′-AACAGCTATGACCATG) (SEQ ID NO:3) and fdSeq (5′-GAATTTTCTGTATGAGG) (SEQ ID NO:2) in a sequence reaction with the Taq sequencing kit with the following cycling protocol: 96° C. for 30 seconds denaturing, 50° C. for 15 seconds annealing and 60° C. for four minutes extension. Precipitated DNA was dissolved in sample buffer, run and analyzed on an ABIPRISM automated fluorescent sequencer. Sequences were compared to the VBASE database and the gene family of each individual chain could be determined.

4. Reactivity of MATDC 16 with Cells in Peripheral Blood

The binding of MatDC16 to subpopulations of PBMC was assessed by triple-staining experiments with FITC-labeled CD14 and CD16, PECy5-labeled CD33 monoclonal antibodies and PE-labeled MoPhabs. For each experiment, 10³ cells within the CD14⁺ CD16⁻CD33⁺ monocyte gate, the CD14⁻CD16⁻CD33⁻ mature DC and the CD14⁻CD16⁻CD33^{dim} precursor DC gates were analyzed. In addition, we performed double-staining experiments with MatDC16 and fluorochrome-labeled lineage-specific monoclonal antibodies including CD3 (T-lymphocytes), CD19 (B-lymphocytes) and CD56 (natural killer cells). Binding of MatDC16 to granulocytes was analyzed based on forward and side scatter profile. As a negative control in staining experiments, a MoPhab specific for thyroglobuline (de Kruif et al., 1995b) was used. MatDC16 brightly stained mature DC, but only a subpopulation of the precursor DCs. It also recognized the CD14⁺ CD16⁻ CD33⁻ blood monocytes. No binding to blood CD3⁺T-cells or CD56⁺ NK-cells was observed for MatDC 16, whereas it did bind to CD19⁺ B-cells.

5. Reactivity of MATDC16 with Tonsil Mononuclear Cells

Human tonsils contain DCs that can be identified as a CD3⁻CD4⁻ cell population that lacks lineage-specific markers. A further division of this population is obtained by staining with antibodies to CDw123. Germinal center DCs, which consist of 65% of the CD3⁻CD4⁺ DCs, are only weakly stained with this antibody (Grouard et al., 1996), whereas the remaining CD3⁻-CD4⁺ DC highly express this marker. Staining of tonsil cell with APC-labeled CD4, PE-labeled CDw123 and FITC-labeled CD3, in combination with indirectly PerCP-labeled MatDC16, was used to examine the reactivity with the different DC populations in tonsil (Table 1). MatDC16 stained the CDw123⁻ DC and the germinal center DCs. No T-cells were recognized. Triple-staining with antibodies specific for IgD and CD38 (Pascual et al., 1994) and MatDC16, revealed that MatDC16 stained the IgD+CD38⁻ naive B-cells, the IgD-CD38+germinal center B-cells and the IgD⁻CD38⁺⁺ plasma blasts. However, no staining of the IgD-CD38⁻ memory B-cells was observed (results not shown).

6. Reactivity of MATDC16 with Hematopoietic Progenitor Cells

In adult bone marrow cells, MatDC16 weakly stained CD34⁺ hematopoietic progenitor cells. It recognized the CD15⁺CD14⁻ myeloid progenitor cells, but not the CD19⁻ B-lymphoid cells and CD3⁺ T-lymphocytes (Table 1).

7. Reactivity of MATDC16 with Synovial Fluid Mononuclear Cells of Patients with Rheumatoid Arthritis

Synovial fluid (SF) from affected joints of patients with rheumatoid arthritis have been shown to contain increased numbers of DCs that may be involved in the prolongation and/or exacerbation of local immune-based inflammatory reactions (Thomas and Quinn, 1996; Hart, 1997). DCs and monocytes in SF may be identified based on the same characteristics as DCs in peripheral blood. MatDC16 stained the mature DCs in SF, whereas a subpopulation of the precursor DCs was also positive (Table 1).

TABLE 1
Reactivity of MoPhabs with different cell populations

| | MatDC11 | MatDC16 | MatDC27 | MatDC51 | MatDC64 |
|---|---|---|---|---|---|
| PBMC: | | | | | |
| Mature DC | +++ | ++ | + | ++/+++ | + |
| Precursor | ++/+++ | ++* | +/++* | ++* | ++* |
| DC | | | | | |
| Monocytes | ++ | ++ | ++ | ++ | ++ |
| CD16⁺ | −/+ | −/+ | −/+ | ++/+++ | −/+ |
| Monocytes | | | | | |
| CD3⁺ T-cells | − | − | − | − | − |
| CD19⁺ B- | + | + | − | − | − |
| cells | | | | | |
| CD56⁺ NK- | − | − | − | − | − |
| cells | | | | | |
| Granulo- | +^{#} | +^{#} | − | + | − |
| cytes: | | | | | |
| Tonsil: | | | | | |
| CDw123^{dim} | ++ | +/++ | −(+) | ++ | −(+) |
| DC | | | | | |
| CDw123⁻ | ++ | ++ | + | −/+ | + |
| DC | | | | | |
| CD3⁺ CD4⁻ | − | − | − | − | − |
| CD3⁻ CD4⁻ | − | − | − | − | − |
| CD3⁻ CD4⁻ | + | + | − | − | − |
| Synovial | | | | | |
| Fluid: | | | | | |
| Mature DC | + | + | − | −/+ | − |
| Precursor | +* | +* | − | −(+) | − |
| DC | | | | | |
| Monocytes | + | + | + | + | + |
| CD33⁻ | − | − | − | − | − |
| CD14⁻ | | | | | |
| cells | | | | | |
| Adult BM: | | | | | |
| CD3⁺ cells | − | − | − | − | − |
| CD10⁺ cells | + | − | − | − | − |
| CD14⁺ cells | + | + | + | + | + |
| CD15⁺ cells | + | −/+ | − | −/+ | − |
| CD19⁺ cells | + | − | − | − | − |
| CD34⁺ cells | + | −/+ | − | −/+ | − |
| Mean fluorescence intensity (MFI) levels for the different populations are shown as −, indicating the highest MFI in the first decade on a four log scale which corresponds to negative control levels. The +, ++ and +++ indicate MFI in the second, third and fourth decades, respectively. A slash (/) indicates that the MFI is on the border between two decades. Parentheses | | | | | |
| # indicate that less than 5% of the population is positive. | | | | | |

*Approximately 50% of the population is positive for this MoPhab.
^{#}10-15% of the granulocytes is positive for this MoPhab.

8. Construction of a Vector for the Production of a Human IgG4-MAGE-1 Fusion Protein

To evaluate the value of antibody-mediated targeted delivery of tumor antigen to DC, a fusion protein was constructed using the constant region of the heavy (H) chain of the human IgG4 gene and the entire coding region of the MAGE-1 molecule. The IgG4 isotype was chosen for this approach since it has low affinity for Fc gamma R1 and does not bind other Fc gamma receptors. In addition, it does not activate the complement system.

Specifically, the Cγ4 genomic DNA was amplified by PCR from vector pNUTCγ4 containing this gene using a 5′ primer containing a BamHI and a NotI site and a 3′ primer containing a SmaI site and a Tyr codon instead of the Cγ4 stopcodon. The amplified Cγ4 DNA was digested with BamHI and SmaI and cloned into the corresponding sites of pNUT, resulting in vector pNUT-Cγ4 without the stopcodon. A SmaI-SmaI cDNA fragment encoding MAGE-1 was fused in-frame at the 3′ terminus of the modified Cγ4 gene. A BamHI-EcOR1 fragment containing the Cγ4-MAGE-1 sequences was removed from the pNUT vector and ligated into pCDNA3.1ΔN+zeo from which, by site-directed mutagenesis, the NotI site in the multiple cloning site was removed.
pHENMatDC16 was digested with NcoI and XhoI to obtain the VH region of MatDC16. Plasmid pLeader was digested with NcoI and SalI, into which sites the VH region was ligated. Then, pLeader-MatDC16 VH was digested with BamHI and NotI, releasing a fragment containing the eukaryotic leader HAVT20 and MatDC16 VH and a donor splice site, which could be cloned into the BamHI and NotI sites of the eukaryotic expression vector pCDNA3.1 ΔN-Cγ4-MAGE-1.
   9. Construction of a Vector for The Production of a Human IgG4-GP100 Fusion Protein

To allow the possibility of directional cloning of any antigen of interest instead of MAGE in plasmid pCDNA3.1-MatDC16-Cγ4-MAGE-1 (SEQ ID NO:14), the SmaI site 5′ of the MAGE gene was changed into a ClaI site by site-directed mutagenesis. The melanoma-specific tumor antigen GP100 was PCR amplified with primers containing a ClaI and a SmaI site, respectively. This PCR fragment was cloned into pTOPO, sequenced and a correct clone was digested with ClaI and SmaI and ligated into ClaI- and SmaI-digested pCDNA3.1-MatDC16-Cγ4, producing plasmid pCDNA3.1-MatDC16-Cγ4-GP100. Production and purification is similar as described below for pCDNA3.1-MatDC16-Cγ4-MAGE.

10. Construction of a Vector for the Production of a Human scFv-MatDC16 Protein

A convenient and powerful expression vector has been developed for Pichia Pastoris. pPicZaB contains Zeocin as a selection marker for cloning both in yeast and bacteria. Heterologous expression of protein is driven by the methanol-inducible promoter for alcohol oxidase AOX1. When methanol is substituted as a carbon source, alcohol oxidase can contribute as much as 30% to the total protein produced, indicating the strength of AOX1 as a promoter. In addition, the expression vector contains the a vector mating sequence to facilitate protein secretion into the medium. At the point of secretion, the signal sequence is cleaved from the expressed protein by the enzyme KEK2 (see SEQ ID NO:19).

Two major changes have been carried out on the basic expression vector:

1. An NcoI-cloning site was introduced immediately after the cleavage point of the secretion-signal peptide (pPicZFVH). With this modification, a simple NcoI-NotI cloning of any scFv into the expression vector will result in expression and secretion of the scFv with its native N-terminus.

2. Vectors have been constructed to allow convenient insertion of fusion partners at the carboxyl tail of the scFv using the NotI and XbaI sites of the multiple cloning site in the vector (pPicZFVH-MAGE-A1, pPicZFVH-gp100).

A region from Gp100 encompassing the immunodominant epitope has been amplified by PCR with primers containing the restriction sites NotI at the N-terminus and XbaI at the C-terminus. The GplO100 fragment was cloned into the vector pPicZFVH containing the scFv MatDC16 (pPicZFVH-MatDC16—Gp100). In the vector, the scFv MatDC16 can be easily exchanged with other scFvs for analysis.

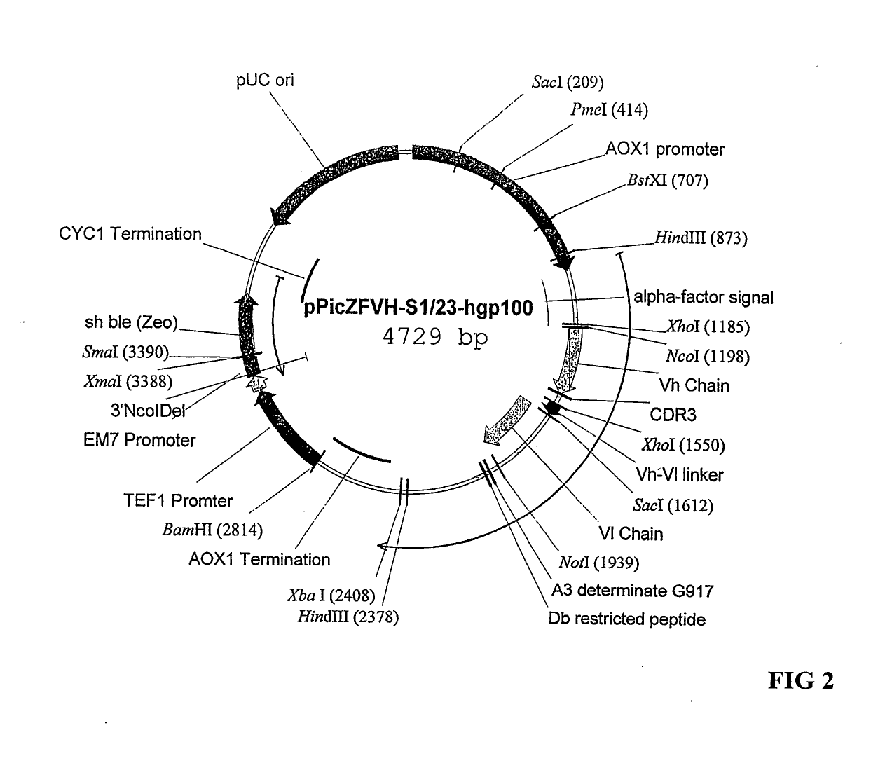

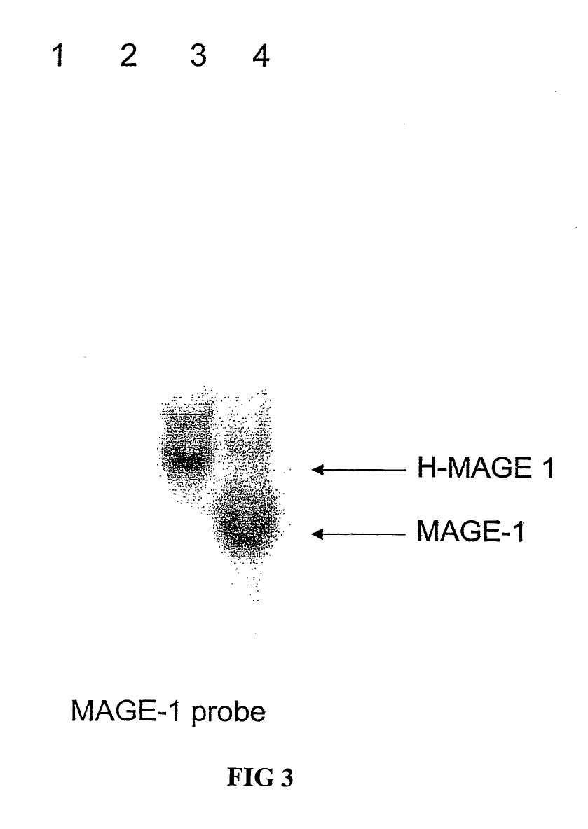

MAGE A1 has been amplified by PCR with primers containing the restriction sites NotI at the N-terminus and XbaI at the C-terminus. The MAGE gene was cloned into the NotI and XbaI site of the vector pPicZFVH containing the scFv MatDC16 (FIG. 2).

11. Construction of a Vector for the Production of a Human scFv with a Chemically Conjugated Plasmid Encoding MAGE

Alternative to producing a fusion protein of antibody and antigen, the antigen can be coupled to the antibody in the form of a DNA plasmid encoding a viral or tumor-derived antigen. The DNA plasmid needs to be condensed in order to get efficient uptake into the target cell. Therefore, polymers such as poly-L-lysine(PLL) and polyethylenimine (PEI) are used. Since coupling of a polymer to the N-terminus of a scFv potentially disrupts its binding capacity, a modified pPicZFHV/MatDC16 construct was prepared. This modified construct encodes the MatDC16 scFv with an additional cysteine residue in front of the stopcodon, resulting in a C-terminal cysteine residue. This modified scFv is produced as described before and used in subsequent coupling reactions.

A coupling reaction involves the following steps:
1. coupling of the heterofunctional cross-linker SMCC to the amine groups of PLL or PEI;
2. purification of the coupled PLL/PEI-SMCC;
3. coupling of the scFv-Cys to PLL/PEI-SMCC via reactivity of the cysteine to the maleimide group of SMCC.

As a second approach, complete antibodies are produced as described for MatDC16-Ig4. These antibodies can be coupled via the N-terminus to the cross-linker SPDP, which contains an internal thiol group. After de-protection, this group can react with the maleimide group in PLL/PEI-SMCC.

12. Transfection and Expression of Antibody-Antigen 1N HEK293 T-Cells

Co-transfection of pCDNA3.1-Cγ4-MAGE-A1 and a construct containing the appropriate immunoglobulin light (L) chain (Boel et al., 2000) into a human cell line resulted in the production of a complete human antibody with the MAGE-1 protein fused to the C-terminus of the heavy chain.

In total, seven different constructs were generated, each construct containing a different V_{H}, resulting in a different antibody specificity (Table 2).

TABLE 2
MoPhabs used to construct fusion proteins: relevant antibody
specificities as determined by flow cytometry are summarized.

| MoPhabs | Specificity |
|---|---|
| MatDC16 | Blood DC, monocytes, immature and mature monocyte- |
| | derived DC |
| MatDC27 | Blood DC, monocytes, immature monocyte-derived DC |
| MatDC64 | Blood DC, monocytes, |
| TN141 | Blood DC, monocytes (weak) |
| 3i-39 | immature monocyte-derived DC |
| MONO14 | Monocytes |
| UBS54 | Epithelial cells, colon carcinoma |

TN141, 3i-39, Monol4 and UBS54 are MoPhabs obtained in other experiments where other cells, DC or colon tumor cells were used as target cells for phage selections.

13. Stable Transfection

To produce whole immunoglobulin fused to MAGE-1, stable transfected cell lines were established by co-transfection of pCDNA3.1-IgG-Cγ4-MAGE-1 including a V_{H} construct, as indicated in Table 2, with the corresponding L-chain construct in HEK293 cells. For transfections, HEK293, a human embryonic kidney cell line, was chosen since correct folding and glycosylation can be anticipated. 1.5×10⁵ HEK293 cells were seeded per well in a 6-well plate. The next day, transfections were carried out at a cell density of 70-80% confluence using calcium chloride precipitated DNA for five hours at 37° C., followed by a 15% glycerol shock for one minute. Five jig of pCDNA3.1-Cγ4-MAGE-1 and 5 μg of the appropriate light chain were used. Cells were washed and after 48 hours 500 μg/ml zeocin was added as selective drug to obtain stable transfectants.

When drug-resistant colonies were large enough, 48 individual clones were picked and expanded.

Later, new transfections have been carried out of the constructs into human Per-C6 cells to produce human antibodies in this preferred cell line.

14. Production and Analysis of IgG4-MAGE-1 Fusion Antibodies

Supernatants from 30 stable clones per construct were screened for production of fusion antibody by a sandwich ELISA. A coating with a mouse-MAGE-1 MoAb was used, supernatant was added and the presence of produced IgG4-MAGE-1 was detected with an HRP-labeled goat-anti-human IgG. From these studies, several clones were selected based on production and the best producing clone, as determined by ELISA, was used for large-scale production in triple-flasks using ULTRA-CHO medium. Three hundred ml culture supernatant was harvested after four days of production, 300 ml fresh ULTRA-CHO was added to the triple-flasks. This procedure was repeated once more. Recombinant protein was purified from 900 ml pooled supernatant using a protein-A column.

15. Characterization of the Recombinant Fusion Antibodies

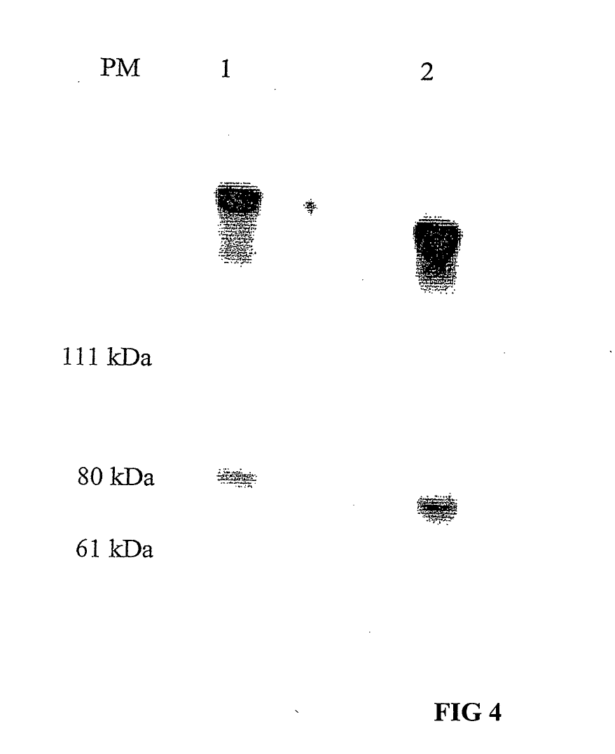

The purified fusion proteins were further characterized by SDS-PAGE and immunoblotting. Under non-reducing conditions, the fusion proteins migrated at an estimated molecular mass of 235 kDa, indicating that the IgG4-MAGE-1 was expressed as a complete antibody-MAGE conjugate (FIG. 4, lane 1). IgG4 (lane 2) can also be seen, due to cross-reactivity of the secondary antibody RAMPO. A faint band of 90 kDa is most probably a partial degradation product (Boel et al., 2000). Under reducing conditions, bands of 100 kDa and 30 kDa can be seen, representing the H-MAGE-1 fusion protein and the L-chain, respectively.

16. FACS Analysis with Recombinant Antibody-Antigen Fusion

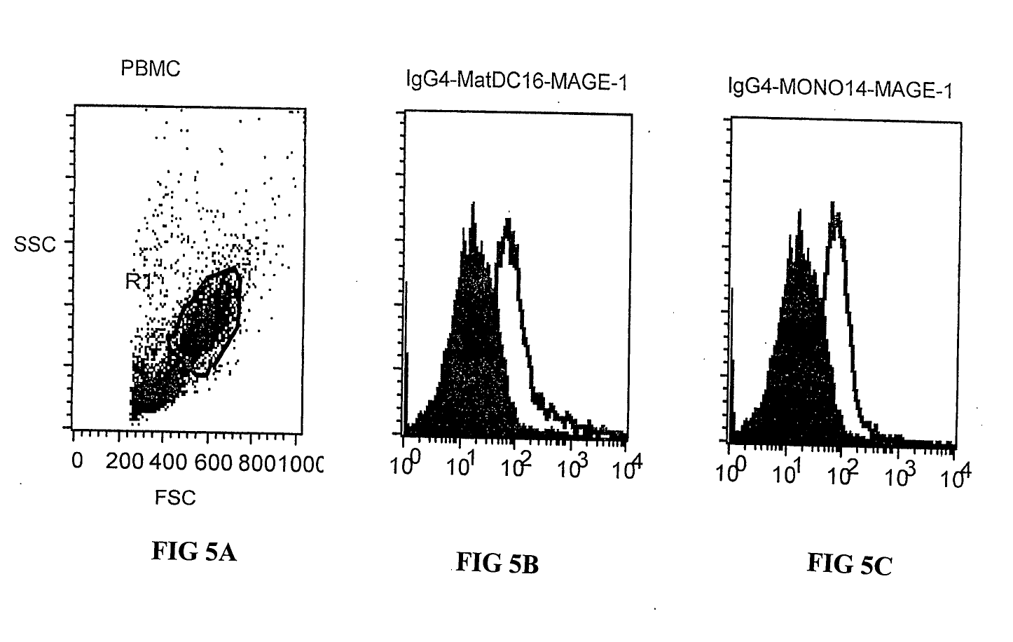

Whether antibody specificity of the different IgG4-MAGE-A1 fusion Moabs was retained, was determined by flow cytometry. Surface binding of the fusion protein to these cells was detected by incubating the cells on ice for 60 minutes with the fusion antibodies. Subsequently, after washing, the cells were incubated with a mouse anti-MAGE-A1 MoAb for 30 minutes. After washing, this step was followed by incubating the cells with PE-conjugated goat anti-mouse Ig as secondary reagent. This assay detects both ends of the fusion protein and, therefore, will detect intact IgG4-MAGE-A1. With flow-cytometric analysis on a FACS Calibur, it was demonstrated that the specificities of the tested fusion antibodies were retained (FIG. 5).

TABLE 3
Reactivity of the IgG4-MAGE-1 fusion proteins on different cells.

| IgG4-MAGE-1 | Monocytes | Immature DC | LS174T |
|---|---|---|---|
| MatDC16 | + | + | − |
| MONO14 | + | n.t. | − |
| UBS54 | − | n.t. | + |

*n.t. = not tested

17. Demonstration of MHC Class I and II Presentation of MAGE-A1 Peptides by Immature DC Targeted with IgG4-MAGE-A1

Next, it was determined whether the IgG4-MAGE-1 fusion antibodies could serve as a source of antigen for immature DC, resulting in presentation via MHC class I and II. Initial experiments were carried out with MatDC16-MAGE-1 that recognizes cultured immature monocyte-derived DC. This antibody also recognizes the immature Mo-DC. As negative controls, MatDC16, without MAGE-1, and UBS54-MAGE-1, which does not recognize immature DC, were used. In addition, IgG4 MONO14-MAGE-1, a fusion antibody that binds to CD14, was included.

Immature monocyte-derived DC were cultured using IL-4 and GM-CSF following standard procedures from HLA.A1/DR1301+donors. Immature DC were incubated with the fusion antibodies (10 μM or 100 nM) or control protein MAGE-A1 (222 nM) and cultured for 24 hours or 48 hours. Subsequently, the DC were replated and cocultured with different T-cell clones.

Activation was assessed as-IFN-gamma release in 24-hour supernatants. Immature DC or CTL alone did not secrete detectable amounts of IFN-gamma (<80 pg/ml/24 hour). The stimulatory capacity of the T-cell clones was assured by exogenous peptide pulsing of the DC with either a MAGE-1.A1-specific peptide, EADPTGHSY (SEQ ID NO:4), in case of the CTL clone, or a MAGE-1.DR13-specific peptide, LLKYRAEPVTKAE (SEQ ID NO:5), in case of the T_{H} clone (data not shown).

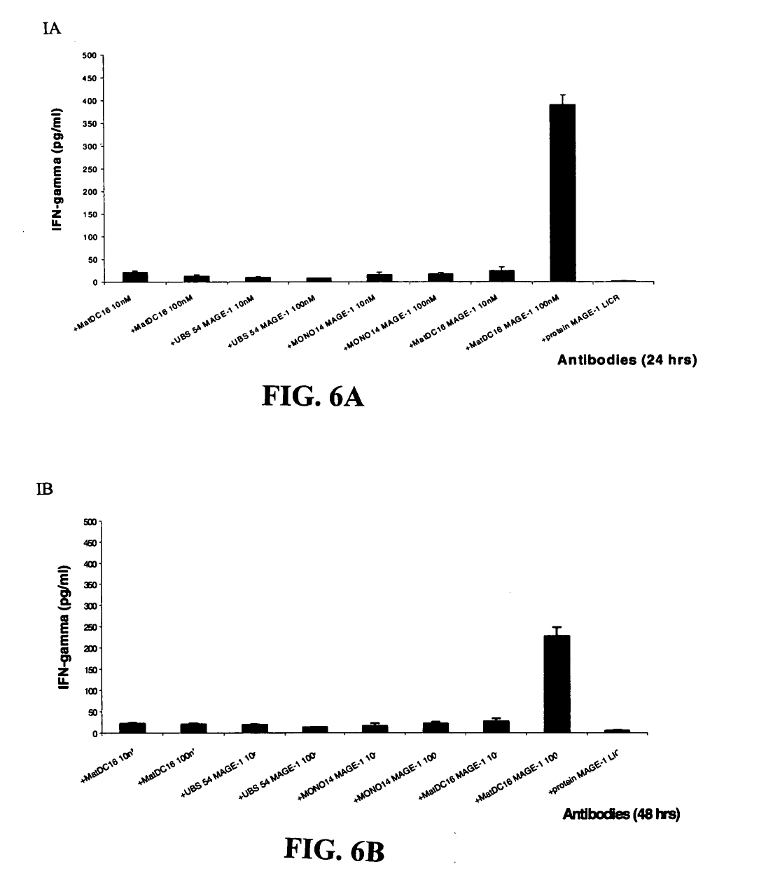

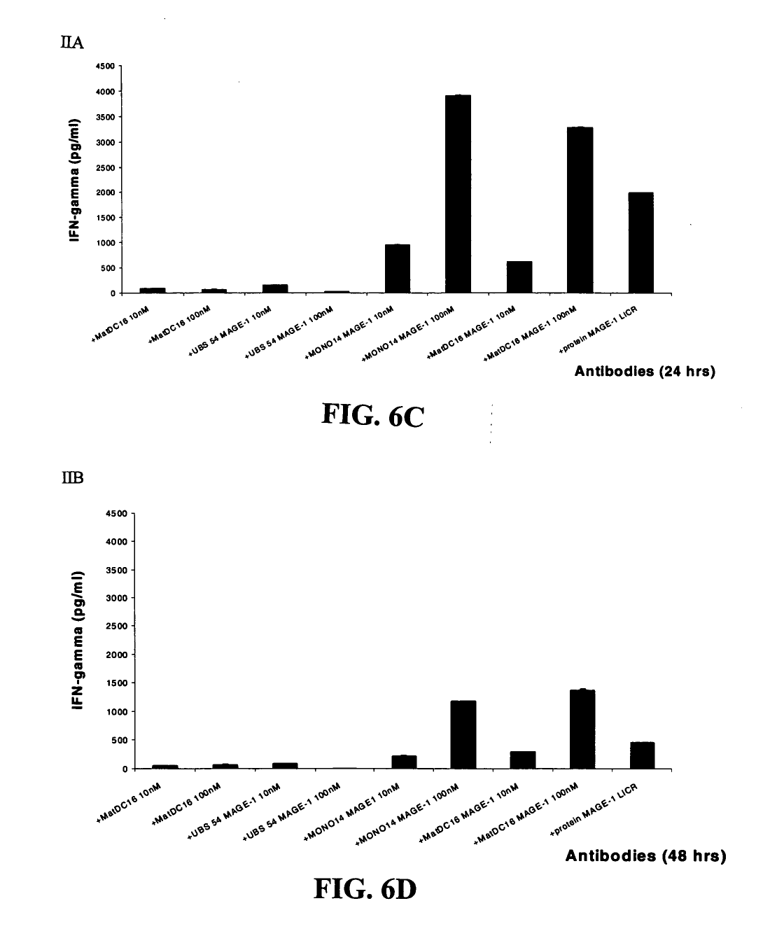

As can be seen in FIG. 6, 100 nM IgGMatDC16-MAGE-1 targeted to immature DC was enough to stimulate a response from the CTL, resulting in a significant amount of IFN-gamma production. No stimulatory activity can be seen for the negative control proteins MatDC16, UBS54-MAGE-1 and MAGE-1. This excludes the possibilities that the response is a consequence of targeting via Fc-gamma-R1 (Wallace et al., 2001) or by pinocytosis. Two different donors for generation of immature DC were used in case of the CTL read-out, giving similar results.

Upon addition of the MAGE-1 protein to immature DC, IFN-g was also produced by the T_{H} clone. This is most likely the result of pinocytosis by the DC, ensuing in MHC class II presentation. Still, targeted delivery of MAGE-1 resulted in an almost two-fold up-regulation of the IFN-g production by the TH clone, demonstrating the efficacy of this approach. The effect seen with the IgG4 MONO14-MAGE-1 may be caused by residual expression of CD14 on the immature DC or additional monocytes in the culture. It is clear that Mono-14 MAGE-1 targeting to immature DCs only results in a TH activation and not in a CTL response. Mono-14 was obtained by phage selections on the CD14⁺ CD33⁺ monocyte population and recognizes the CD14 molecule as determined by specific staining of CHO cells transfected with the human CD14 cDNA (results not shown). Taken together, these initial data demonstrate a very efficient induction of dual MAGE-A1 responses, using MatDC16 IgG4-MAGE-A1 fusion antibody targeted to DC.

### REFERENCES

1. Wallace P. K., Tsang K. Y., Goldstein J., Correale P., Jarry T. M., Schlom J., Guyre P. M., Emstoff M. S., Fanger M. W. Exogenous antigen targeted to FcgammaRI on myeloid cells is presented in association with MHC class I. J. Immunol. Methods. 2001 Feb. 1; 248(1-2): 183-94.
2. Amigorena S., Bonnerot C. Fc receptors for IgG and antigen presentation on MHC class I and class II molecules. Semin. Immunol. 1999 December; 11(6):385-90.
3. Machy P., Serre K., Leserman L. Class I-restricted presentation of exogenous antigen acquired by Fcgamma receptor-mediated endocytosis is regulated in dendritic cells. Eur. J. Immunol. 2000 March; 30(3):848-57.
4. Lekkerkerker A., Logtenberg T. Phage antibodies against human dendritic cell subpopulations obtained by flow cytometry-based selection on freshly isolated cells. J. Immunol. Methods. 1999 Dec. 10; 231(1-2):53-63.
5. Thomas R., Davis L. S., Lipsky P. E. Isolation and characterization of human peripheral blood dendritic cells. J. Immunol. 1993 Feb. 1; 150(3):821-34.
6. Thomas R., Lipsky P. E. Human peripheral blood dendritic cell subsets. Isolation and characterization of precursor and mature antigen-presenting cells. J. Immunol. 1994 Nov. 1; 153(9):4016-28.
7. O'Doherty U., Peng M., Gezelter S., Swiggard W. J., Betjes M., Bhardwaj N., Steinman R. M. Human blood contains two subsets of dendritic cells, one immunologically mature and the other immature. Immunology. 1994 July; 82(3):487-93.
8. Olweus J., BitMansour A., Warnke R., Thompson P. A., Carballido J., Picker L. J., Lund-Johansen F. Dendritic cell ontogeny: a human dendritic cell lineage of myeloid origin. Proc. Natl. Acad. Sci. U.S.A. 1997 Nov. 11; 94(23):12551-6.
9. Grouard G., Rissoan M. C., Filgueira L., Durand I., Banchereau J., Liu Y. J. The enigmatic plasmacytoid T-cells develop into dendritic cells with interleukin (IL)-3 and CD40-ligand. J. Exp. Med. 1997 Mar. 17; 185(6):1101-11.
10. Grouard G., Durand I., Filgueira L., Banchereau J., Liu Y. J. Dendritic cells capable of stimulating T-cells in germinal centres. Nature. 1996 Nov. 28; 384(6607):364-7.
11. Rosenberg S. A., Yang J. C., Schwartzentruber D. J., Hwu P., Marincola F. M., Topalian S. L., Restifo N. P., Dudley M. E., Schwarz S. L., Spiess P. J., Wunderlich J. R., Parkhurst M. R., Kawakami Y., Seipp C. A., Einhom J. H., White D. E. Immunologic and therapeutic evaluation of a synthetic peptide vaccine for the treatment of patients with metastatic melanoma. Nat. Med. 1998 March; 4(3):321-7.
12. Rosenberg S. A. A new era for cancer immunotherapy based on the genes that encode cancer antigens. Immunity. 1999 March; 10(3):281-7.
13. Nestle F. O., Burg G., Dummer R. New perspectives on immunobiology and immunotherapy of melanoma. Immunol. Today. 1999 January; 20(1):5-7.
14. Boon T., van der Bruggen P. Human tumor antigens recognized by T-lymphocytes. J. Exp. Med. 1996 Mar. 1; 183(3):725-9.
15. van der Bruggen P., Traversari C., Chomez P., Lurquin C., De Plaen E., Van den Eynde B., Knuth A., Boon T. A gene encoding an antigen recognized by cytolytic T-lymphocytes on a human melanoma. Science. 1991 Dec. 13; 254 (5038):1643-7.
16. Lanzavecchia A., Sallusto F. Antigen decoding by T-lymphocytes from synapses to fate determination. Nat. Immunol. 2001 June; 2(6):487-92.
17. de Kruif J., Boel E., Logtenberg T. Selection and application of human single chain Fv antibody fragments from a semi-synthetic phage antibody display library with designed CDR3 regions. J. Mol. Biol. 1995 Apr. 21; 248(1):97-105.
18. Pascual V., Liu Y. J., Magalski A., de Bouteiller O., Banchereau J., Capra J. D. Analysis of somatic mutation in five B-cell subsets of human tonsil. J. Exp. Med. 1994 Jul. 1; 180(1):329-39.
19. Thomas R., Quinn C. Functional differentiation of dendritic cells in rheumatoid arthritis: role of CD86 in the synovium. J. Immunol. 1996 Apr. 15; 156(8):3074-86.
20. Hart D. N. Dendritic cells: unique leukocyte populations which control the primary immune response. 1997 Nov. 1; 90(9):3245-87.
21. Boel E., Verlaan S., Poppelier M. J., Westerdaal N. A., Van Strijp J. A., Logtenberg T. Functional human monoclonal antibodies of all isotypes constructed from phage display library-derived single-chain Fv antibody fragments. J. Immunol. Methods. 2000 May 26; 239(1-2):153-66.

1. A conjugate for targeting an antigen-presenting cell comprising:
at least one antigenic moiety conjugated to a targeting moiety to form a conjugate that is capable of binding to a cell surface structure of an antigen-presenting cell, and, upon binding, inducing a cytotoxic T-cell type response and a T-helper response.

2. The conjugate of claim 1, wherein the antigen-presenting cell presents at least a fragment of the at least one antigenic moiety via a major histocompatibility complex (MHC) class I and a MHC class II molecule on a surface of the antigen-presenting cell, wherein the at least a fragment of the at least one antigenic moiety is produced within the antigen-presenting cell.

3. The conjugate of claim 2, wherein the targeting moiety is selected from the group consisting of an immunoglobulin or fragment thereof, wherein the fragment comprises a scFv fragment, a Fab fragment, or a F(ab)₂ fragment.

4. The conjugate of claim 3, wherein the targeting moiety is a monoclonal antibody or fragment thereof.

5. The conjugate of claim 3, wherein the targeting moiety is humanized or human.

6. The conjugate of claim 2, wherein the targeting moiety is bivalent or polyvalent.

7. The conjugate of claim 2, wherein the antigen-presenting cell comprises a B cell, a monocyte, or a dendritic cell.

8. The conjugate of claim 7, wherein the targeting moiety recognizes CD33⁺ CD14⁻ dendritic cells and CD33^{dim} CD16⁻ dendritic cells, and which essentially does not recognize CD3⁺ T cells and CD56⁺ NK cells.

9. The conjugate of claim 8, wherein the targeting moiety is MatDC16 (SEQ ID NO:21), in the strain deposited at the ECACC under accession number 01120417.

10. The conjugate of claim 2, wherein the at least one antigenic moiety is of parasitic, fungal, bacterial, viral or autologous origin.

11. The conjugate of claim 2, wherein the at least one antigenic moiety is selected from the group consisting of a peptide, polypeptide, protein, glycoprotein, lipoprotein, and a fragment thereof.

12. The conjugate of claim 11, wherein at least one antigenic moiety is selected from the group of antigens consisting of MAGE, gp100, gag, env, MUC, PAGE, CEA, PSA, PSMA, and combinations thereof.

13. The conjugate of claim 2, wherein the antigenic moiety comprises a nucleic acid sequence encoding a peptide.

14. The conjugate of claim 13, wherein the nucleic acid is a mammalian expression vector.

15. The conjugate of claim 7, wherein the targeting moiety is selected from the group of targeting moieties consisting of MatDC11, MatDC27, MatDC51, and MatDC64.

16. An isolated nucleic acid sequence comprising a nucleic acid sequence encoding a fusion peptide comprising an antigenic moiety and a targeting moiety, wherein the targeting moiety recognizes a cell surface structure of an antigen-presenting cell, and wherein upon binding, the antigenic moiety induces a cytotoxic T-cell type response and a T-helper response.

17. The isolated nucleic acid sequence of claim 16, wherein the nucleic acid sequence is operably linked to an expression sequence recognized by an antigen-presenting cell.

18. The isolated nucleic acid sequence of claim 17, wherein the expression sequence comprises a hCMV promoter.

19. The isolated nucleic acid sequence of claim 18, further comprising a polyA signal from bovine growth hormone.

20. The isolated nucleic acid sequence of claim 16, wherein the targeting moiety comprises an immunoglobulin or fragment thereof, wherein the fragment comprises a scFv fragment, a Fab fragment, or a F(ab)2 fragment.

21. A host cell transformed or transfected with the isolated nucleic acid sequence of claim 16, wherein the nucleic acid sequence is operably linked to an expression sequence.

22. The host cell of claim 21, wherein the host cell is PER-C6.

23. A method for producing a conjugate, the method comprising:
introducing into a host cell a nucleic acid encoding a conjugate comprising an antigenic moiety and a targeting moiety;
culturing the host cell;
expressing the nucleic acid to produce the conjugate; and
isolating the conjugate.

24. A method for generating an antigen-presenting cell capable of eliciting an immune response via presentation of a processed antigen on a major histocompatibility complex (MHC) class I and MHC class II molecule of the antigen-presenting cell, the method comprising:
contacting an antigen-presenting cell with a conjugate comprising at least one antigenic moiety conjugated to a targeting moiety, wherein the targeting moiety recognizes a cell surface structure on an antigen-presenting cell;
internalizing the conjugate into the antigen-presenting cell;
processing the antigenic moiety to produce a processed antigen; and
presenting the processed antigen on a MHC class I and MHC class II molecule of the antigen-presenting cell.

25. The method according to claim 24, wherein the targeting moiety is selected from the group of targeting moieties consisting of MatDC11, MatDC27, MatDC51, and MatDC64.

26. The method according to claim 24, wherein the targeting moiety is MatDC16 (SEQ ID NO:21).

27. An antigen-presenting cell produced by the method of claim 24.

28. A pharmaceutical composition comprising the antigen-presenting cell of claim 27.

29. A method of producing an antigenic moiety-specific adaptive immune response in a subject, the method comprising:
administering a conjugate comprising an antigenic moiety conjugated to an antigen-presenting cell specific targeting moiety to an antigen-presenting cell;
internalizing the conjugate into the antigen-presenting cell;
processing the antigenic moiety to produce a processed antigen;
presenting the processed antigen on a MHC class I and MHC class II molecule of the antigen-presenting cell; and
producing an antigenic moiety-specific adaptive immune response in a subject.

30. The method according to claim 29, wherein the antigenic moiety is selected from the group consisting of a parasitic antigen, a fungal antigen, a bacterial antigen, a viral antigen and a tumor antigen.

31. The method according to claim 29, administering the conjugate comprises administering the conjugate to the subject.

32. The method according to claim 29, comprising producing an antigenic moiety-specific adaptive immune response to a disease comprising melanoma.

33. The method according to claim 29, comprising selecting the targeting moiety from the group of targeting moieties consisting of MatDC11, MatDC27, MatDC51, and MatDC64.

34. The method according to claim 29, wherein administering a conjugate comprises administering a conjugate wherein the targeting moiety is MatDC16 (SEQ ID NO:21).

35. The method according to claim 29, wherein producing an antigenic moiety-specific adaptive immune response results in vaccinating the subject.

36. The method according to claim 29, comprising inducing a cytotoxic T lymphocyte response and a T-helper response.

37. A conjugate comprising a targeting moiety obtained from MatDC16 (SEQ ID NO:21), as deposited in the strain at the ECACC, under accession number 01120417 conjugated to an antigenic moiety.

38. The conjugate of claim 37, comprising an affinity matured mutant of MatDC16 (SEQ ID NO:21).

39. A pharmaceutical composition comprising a conjugate of claim 2 in a pharmaceutically acceptable form.


SEQUENCE LISTING (21 sequences)

SEQ ID NO: 1 - DNA, 17 bp, Artificial Sequence
  Synthetic oligonucleotide LMB3
caggaaacagctatgac

SEQ ID NO: 2 - DNA, 17 bp, Artificial Sequence
  Synthetic oligonucleotide fd-SEQ1
gaattttctgtatgagg

SEQ ID NO: 3 - DNA, 16 bp, Artificial Sequence
  Synthetic oligonucleotide M13REV
aacagctatgaccatg

SEQ ID NO: 4 - protein, 9 aa, Artificial Sequence
  MAGE-1.A1 specific peptide
EADPTGHSY

SEQ ID NO: 5 - protein, 13 aa, Artificial Sequence
  MAGE-1.DR13 specific peptide
LLKYRAEPVTKAE

SEQ ID NO: 6 - protein, 19 aa, Artificial Sequence
  TcR leader of MatDC16
MACPGFLWALVISTCLEFS

SEQ ID NO: 7 - protein, 32 aa, Artificial Sequence
  FR1 of MatDC16
MAQVQLVQSGAEVKKPGASVKVSCKASGYTFT

SEQ ID NO: 8 - protein, 5 aa, Artificial Sequence
  CDR1 of MatDC16
SYGIS

SEQ ID NO: 9 - protein, 14 aa, FR2 of MatDC16
WVRQAPGQGLEWMG

SEQ ID NO: 10 - protein, 17 aa, Artificial Sequence
  CDR2 of MatDC16
WISAYNGNTNYAQKLQG

SEQ ID NO: 11 - protein, 32 aa, Artificial Sequence
  FR3 of MatDC16
RVTITTDTSTSTAYMELRSLRSDDTAVYYCAR

SEQ ID NO: 12 - protein, 9 aa, Artificial Sequence
  CDR3 of MatDC16
ASLYSKFDY

SEQ ID NO: 13 - protein, 11 aa, Artificial Sequence
  FR4 of MatDC16
WGQGTLVTVST

SEQ ID NO: 14 - protein, 1342 aa, Artificial Sequence
  MatDC16-Cgamma4-MAGE-A1
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGVMAQVQLVQSGAEVKKPGASVKVSCKASGYTFTSYGISWVRQAPGQGLEWMGWISAYNGNTNYAQKLQGRVTITTDTSTSTAYMELRSLRSDDTAVYYCARASLYSKFDYWGQGTLVTVSTGECGREPRHWTLHGPSRIDKNRGASAPWAQLCPTPRSHGTTSLAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRDTHLPGLSYSPSGLYSLSSVVTVPSSSLGTKTYTXNVDHKPSNTKVDKRVGERPAQGGRVSAGSQAQPSCLDRTPAVQPQPRAARHAPSVSSPGGLPPHSCSGRGSSGFFHQAPGSHRLDAPTPGPAHTGAGAALRPAKSHIREDPAPDLSPPQRPNSPLPQLRHLLSSQIVTPNLLSAESKYGPPCPSCPGKPTQASPSSSRRDRCPRVACIQGQAPAGCRIHLHLFLSTVMSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 15 - DNA, 55 bp, Artificial Sequence
  Sense strand of the pPicZ alpha B cloning site
tctctcgagaaaagagaggctgaagctgcaggaattcacgtggcccagccggccg

SEQ ID NO: 16 - DNA, 55 bp, Artificial Sequence
  Antisense strand of the pPicZ alpha B cloning site
agagagctcttttctctccgacttcgacgtccttaagtgcaccgggtcggccggc

SEQ ID NO: 17 - protein, 18 aa, Artificial Sequence
  Peptide translation of pPicZ alpha B cloning site
SLEKREAEAAGIHVAQPA

SEQ ID NO: 18 - DNA, 55 bp, Artificial Sequence
  Sense strand of the pPicZFVH cloning site
tctctcgagaaaagagccatggaagctgcaggaattcacgtggcccagccggccg

SEQ ID NO: 19 - DNA, 55 bp, Artificial Sequence
  Antisense strand of the pPicZFVH cloning site
agagagctcttttctcggtaccttcgacgtccttaagtgcaccgggtcggccggc

SEQ ID NO: 20 - protein, 18 aa, Artificial Sequence
  Peptide translation of pPicZFVH cloning site
SLEKRAMEAAGIHVAQPA

SEQ ID NO: 21 - protein, 1052 aa, Artificial
  MatDC16-Cgamma4-MAGE-1
MACPGFLWALVISTCLEFSMAQVQLVQSGAEVKKPGASVKVSCKASGYTFTSYGISWVRQAPGQGLEWMGWISAYNGNTNYAQKLQGRVTITTDTSTSTAYMELRSLRSDDTAVYYCARASLYSKFDYWGQGTLVTVSTGECGREPRHWTLHGPSRIDKNRGASAPWAQLCPTPRSHGTTSLAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRDTHLPGLSYSPSGLYSLSSVVTVPSSSLGTKTYTXNVDHKPSNTKVDKRVGERPAQGGRVSAGSQAQPSCLDRTPAVQPQPRAARHAPSVSSPGGLPPHSCSGRGSSGFFHQAPGSHRLDAPTPGPAHTGAGAALRPAKSHIREDPAPDLSPPQRPNSPLPQLRHLLSSQIVTPNLLSAESKYGPPCPSCPGKPTQASPSSSRRDRCPRVACIQGQAPAGCRIHLHLFLSTVMSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV